(19) United States
(12) Patent Application Publication
(10) Pub. No.: US 2014/0350255 A1
(43) Pub. Date: Nov. 27, 2014

(54) PROCESS FOR THE PREPARATION OF VILAZODONE OR ITS PHARMACEUTICALLY ACCEPTABLE SALTS

(71) Applicant: RANBAXY LABORATORIES LIMITED, New Delhi, Delhi (IN)

(72) Inventors: Prasenjit Prafulla Das, Nagpur (IN); Bindu Srivastava, North West Delhi (IN); Nitin Maheshwari, New Delhi (IN); Hashim Nizar Poovanathil Nagoor Meeran, Pathanamthitta (IN); Mohan Prasad, Gurgaon (IN); Sudershan Kumar Arora, Gurgaon (IN)

(21) Appl. No.: 14/375,938

(22) PCT Filed: Feb. 1, 2013
(86) PCT No.: PCT/IB2013/050881

§ 371 Date: Jul. 31, 2014

(30) Foreign Application Priority Data
Feb. 1, 2012 (IN) 281/DEL/2012

(51) Int. Cl. C07D 405/12

(52) U.S. Cl. CPC C07D 405/12
USPC 544/373

(57) ABSTRACT

The present invention relates to a process for the preparation of vilazodone or its pharmaceutically acceptable salts. The present invention further provides a crystalline form of vilazodone free base.

### FIELD OF THE INVENTION

The present invention relates to a process for the preparation of vilazodone or its pharmaceutically acceptable salts. The present invention further provides a crystalline form of vilazodone free base.

### BACKGROUND OF THE INVENTION

Vilazodone is chemically described as 5-{4-[4-(5-cyano-1H-indol-3-yl)butyl]piperazin-1-yl}-1-benzofuran-2-carboxamide of Formula I.

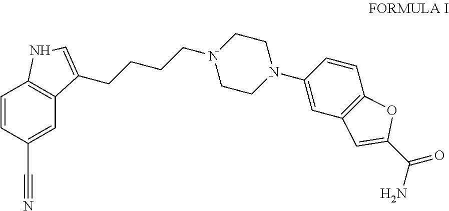 [SMILES: N#Cc1ccc2[nH]cc(CCCCN3CCN(c4ccc5oc(C(N)=O)cc5c4)CC3)c2c1]

Vilazodone is indicated for the treatment of a major depressive disorder (MDD).

Processes for the preparation of vilazodone are described in U.S. Pat. Nos. 5,532,241 and 7,799,916, and European Patent No. EP 0 648 767.

### SUMMARY OF THE INVENTION

The present invention relates to a process for the preparation of vilazodone or its pharmaceutically acceptable salts.

### BRIEF DESCRIPTION OF THE DRAWINGS

FIG. 1 depicts the X-Ray Powder Diffraction Pattern (XRPD) of the vilazodone free base obtained according to Example 1.

FIG. 1A provides the table of values for the XRPD pattern depicted in FIG. 1.

FIG. 2 depicts the X-Ray Powder Diffraction Pattern (XRPD) of the vilazodone free base obtained according to Example 3.

FIG. 2A provides the table of values for the XRPD pattern depicted in FIG. 2.

### DETAILED DESCRIPTION OF THE INVENTION

An aspect of the present invention provides a process for the preparation of 5-{4-[4-(5-cyano-1H-indol-3-yl)butyl]piperazin-1-yl}-1-benzofuran-2-carboxamide of Formula I

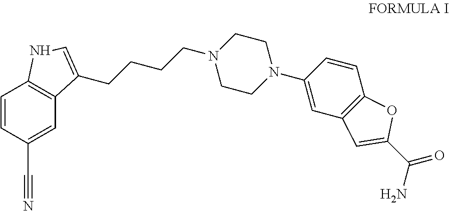 [SMILES: N#Cc1ccc2[nH]cc(CCCCN3CCN(c4ccc5oc(C(N)=O)cc5c4)CC3)c2c1]

or its pharmaceutically acceptable salts which comprises:

a) reacting 3-(4-chlorobutyl)-1H-indole-5-carbonitrile of Formula II

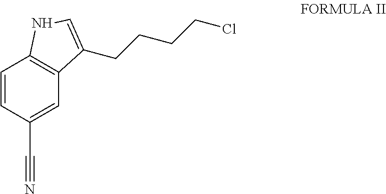 [SMILES: N#Cc1ccc2[nH]cc(CCCCCl)c2c1]

with 5-(piperazin-1-yl)-1-benzofuran-2-carboxamide of Formula III

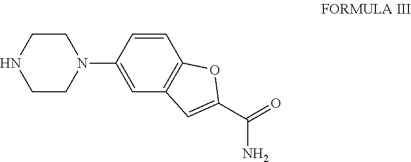 [SMILES: NC(=O)c1cc2cc(N3CCNCC3)ccc2o1]

in the presence of a base and solvent;

b) isolating the compound of Formula I from the reaction mixture thereof; and

c) optionally converting the compound of Formula Ito its pharmaceutically acceptable salts.

Treatment of the compound of Formula II and the compound of Formula III may be carried out in the presence of a base and solvent. The solvent may be selected from a group consisting of water, organic solvent, or a mixture thereof. Suitable organic solvents may be selected from a group consisting of alcohol, ketone, nitrile, amide, aromatic or aliphatic hydrocarbon, or dimethyl sulfoxide. Suitable alcoholic solvents may include methanol, 2-propanol, or 1-propanol. Suitable nitrile solvents may include acetonitrile. Suitable amide solvents may include N-methylpyrrolidone or dimethylformamide. Suitable ketonic solvents may include acetone or methyl isobutyl ketone. Suitable aromatic hydrocarbon solvents may include toluene. Preferable solvents may include water alone or in combination with 2-propanol, 1-propanol, dimethylformamide, or toluene. The treatment of the compound of Formula II and the compound of Formula III may be carried out in the presence of only water without using any other solvent.

The base may be selected from a group consisting of organic base or inorganic base. A suitable organic base may include triethylamine, diisopropylamine, diisopropylethylamine, 4-dimethylaminopyridine, pyrollidine, or N-methyl morpholine. A preferable organic base includes triethylamine A suitable inorganic base may include hydroxides or carbonates and bicarbonates of alkali or alkaline metal. Suitable carbonates or bicarbonates of alkali or alkaline metal may include sodium carbonate, potassium carbonate, magnesium carbonate, sodium bicarbonate, or potassium bicarbonate. A preferable inorganic base includes potassium carbonate. Treatment of the compound of Formula II and the compound of Formula III may be carried out in the presence of alkali metal halides, for example, sodium iodide.

The treatment of the compound of Formula II and the compound of Formula III may be carried out a temperature of about 5° C. to about 110° C., preferably at about 15° C. to about 90° C. The treatment of the compound of Formula II and the compound of Formula III may be carried for about 2 hours to about 35 hours, preferably for about 5 hours to about 30 hours.

The vilazodone free base of Formula I prepared by the present invention may be converted to its pharmaceutically acceptable salt before isolation. The vilazodone free base of Formula I may be isolated by filtration, concentration, precipitation, cooling, centrifugation, decantation, or a combination thereof.

Vilazodone free base obtained by the present invention can be converted to its pharmaceutically acceptable salt, for example hydrochloric acid salt, by any of the method known in the art. The vilazodone hydrochloride salt may be isolated by filtration, decantation, or a combination thereof.

Another aspect of the present invention provides a crystalline form of vilazodone free base.

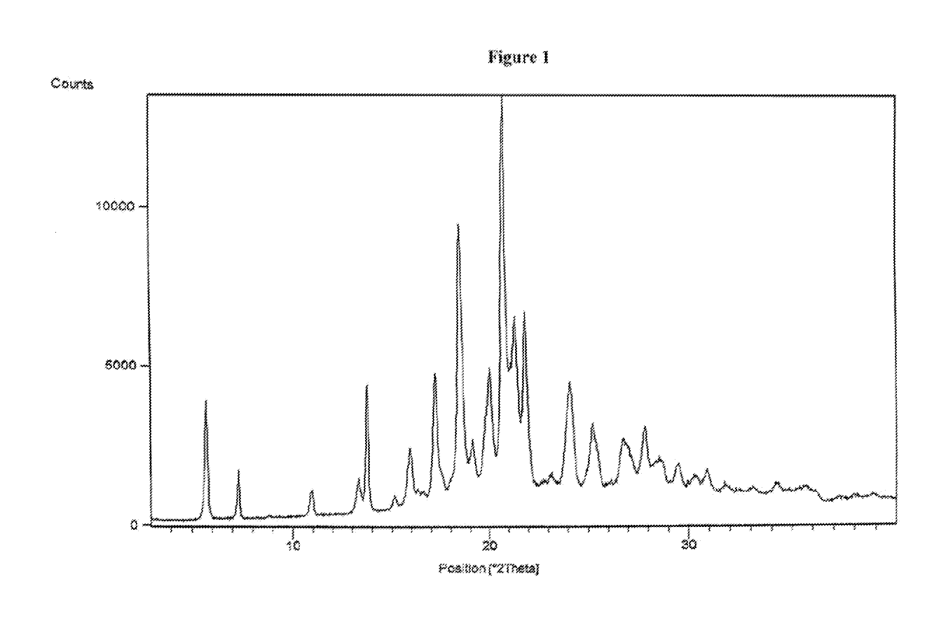

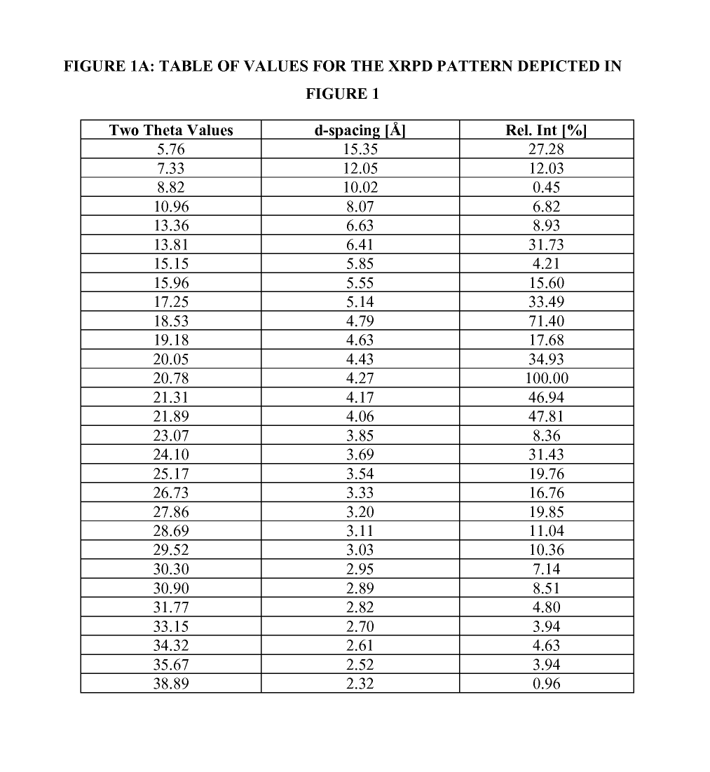

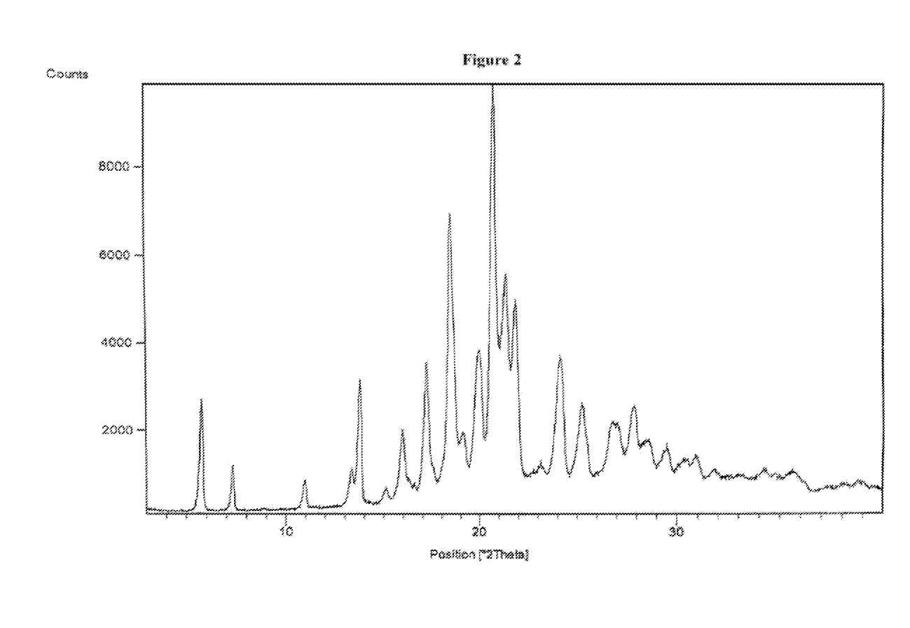

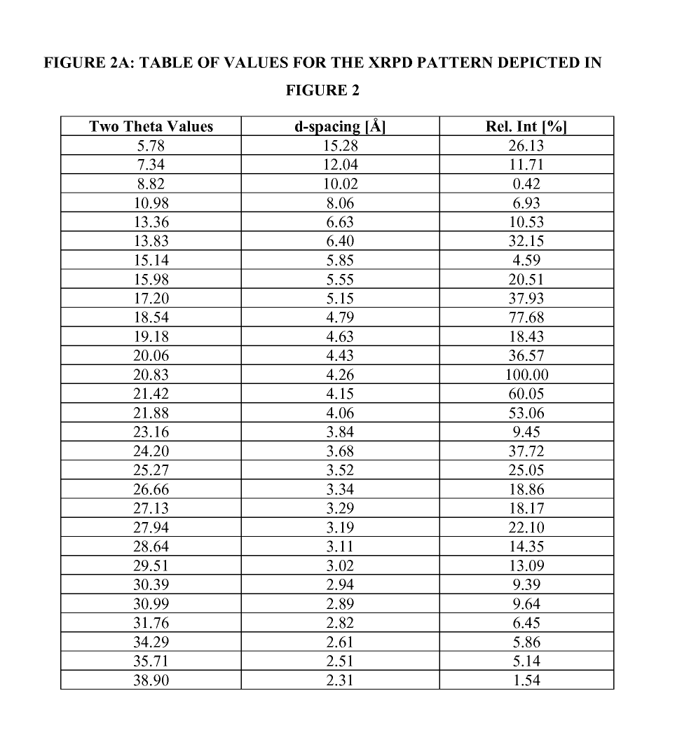

Crystalline form of vilazodone free base has substantially the same XRPD (X-Ray Powder Diffraction Pattern) pattern as depicted in FIG. 1 or FIG. 2. The crystalline form of vilazodone free base is characterized by an XRPD pattern having interplanar spacing (d) values substantially at 6.41, 5.14, 4.79, 4.43, 4.27, 4.17, 4.06, and 3.69±0.2 Å. The crystalline form of vilazodone free base is further characterized by an XRPD pattern having interplanar spacing (d) values substantially at 15.35, 12.05, 10.02, 8.07, 6.63, 6.41, 5.85, 5.55, 5.14, 4.79, 4.63, 4.43, 4.27, 4.17, 4.06, 3.85, 3.69, 3.54, 3.33, 3.20, 3.11, 3.03, 2.95, 2.89, 2.82, 2.70, 2.61, 2.52, and 2.32±0.2 Å.

XRPD of the samples were determined by using Panalytical X'Pert Pro X-Ray Powder Diffractometer in the range 3-40 degree 2 theta and under tube voltage and current of 45 Kv and 40 mA respectively. Copper radiation of wavelength 1.54 angstrom and Xceletor detector were used.

In the following section, embodiments are described by way of examples to illustrate the process of invention. Several variants of these examples would be evident to persons ordinarily skilled in the art.

### EXAMPLE 1

### Preparation of 5-{4-[4-(5-Cyano-1H-Indol-3-yl)Butyl]Piperazin-1-yl}-1-Benzofuran-2-Carboxamide

3-(4-Chlorobutyl)-1H-indole-5-carbonitrile (34.2 g) was added to acetonitrile (300 mL). Sodium iodide (33.1 g) was added to the reaction mixture. The reaction mixture was heated to 80° C. to 85° C. and maintained for 60 minutes. The reaction mixture was cooled to 60° C. 5-(Piperazin-1-yl)-1-benzofuran-2-carboxamide (30 g) and triethylamine (18.6 g) were added to the reaction mixture. The reaction mixture was heated to 80° C. to 83° C. for 18 hours. The reaction mixture was cooled to 20° C. to 30° C. The reaction mixture was added to water (300 mL), dichloromethane (300 mL), and sodium thiosulphate (7.5 g). Concentrated hydrochloric acid (15 mL) was added to the reaction mixture and stirred for 1.5 hours at 20° C. to 30° C. The solid obtained was filtered and washed with dichloromethane (80 mL) and deionized water (150 mL). The reaction mixture was added to ethyl acetate (600 mL) and water (300 mL). Triethyl amine (20 g) was added to the reaction mixture. The reaction mixture was heated to 70° C. to 75° C. The layers obtained were separated and the organic layer was washed with water (100 mL). The organic layer was recovered under vacuum. 2-Propanol (60 mL) was added to the reaction mixture and stirred at 20° C. to 30° C. for 1 hour. The reaction mixture was filtered, washed with 2-propanol (30 mL) and dried under vacuum at 45° C. to 50° C. for 12 hours to obtain the title compound having XRPD as depicted in FIG. 1.

### Yield: 44.0 g.

### EXAMPLE 2

### Preparation of 5-{4-[4-(5-Cyano-1H-Indol-3-yl)Butyl]Piperazin-1-yl}-1-Benzofuran-2-Carboxamide

Potassium carbonate (11.3 g) was added to water (100 mL) and stirred for 10 minutes. 5-Piperazin-1-ylbenzofuran-2-carboxamide (20 g) and 3-(4-chlorobutyl)indole-5-carbonitrile (20.9 g) were added to the reaction mixture. The reaction mixture was heated to 98° C. to 100° C. and maintained for 5 hours. The reaction mixture was cooled to 50° C. Water (200 mL) was added to the reaction mixture. The solid obtained was filtered and dried in an air oven at 50° C. to 55° C. for 10 hours to 12 hours to obtain the title compound.

### Yield: 18.0 g.

### EXAMPLE 3

### Preparation of 5-{4-[4-(5-Cyano-1H-Indol-3-yl)Butyl]Piperazin-1-yl}-1-Benzofuran-2-Carboxamide

Potassium carbonate (28.2 g) was added to water (250 mL) and stirred for 10 minutes. 5-Piperazin-1-ylbenzofuran-2-carboxamide (50 g), 3-(4-chlorobutyl)indole-5-carbonitrile (52.3 g), and 2-propanol (250 mL) were added to the reaction mixture. The reaction mixture was heated to 80° C. to 85° C. and maintained for 24 hours. The reaction mixture was cooled to 30° C. Water (500 mL), dichloromethane (500 mL), and hydrochloric acid (3N; 200 mL) were added to the reaction mixture and filtered. The solid obtained was dissolved in ethyl acetate (1000 mL), triethylamine (50 mL), and water (100 mL) at 80° C. to 83° C. The reaction mixture was filtered and the organic layer was separated and recovered. The residue was treated with 2-propanol (150 mL) and filtered. The solid obtained was dried under vacuum at 45° C. to 50° C. to obtain the title compound having XRPD as depicted in FIG. 2.

### Yield: 57.5 g.

### EXAMPLE 4

### Preparation of 5-{4-[4-(5-Cyano-1H-Indol-3-yl)Butyl]Piperazin-1-yl}-1-Benzofuran-2-Carboxamide

Potassium carbonate (28.2 g) was added to water (250 mL) and stirred for 10 minutes. 5-Piperazin-1-ylbenzofuran-2-carboxamide (50 g), 3-(4-chlorobutyl)indole-5-carbonitrile (52.3 g), and 2-propanol (250 mL) were added to the reaction mixture. The reaction mixture was heated to 80° C. to 85° C. and maintained for 24 hours. The reaction mixture was cooled to 25° C. to 30° C. and filtered. The reaction mixture was added to dichloromethane (350 mL), methanol (350 mL), and concentrated hydrochloric acid (25 mL) at 30° C. to 35° C. Water (350 mL) was added to the reaction mixture and the mixture was stirred and filtered. The solid obtained was dissolved in ethyl acetate (1000 mL), methanol (250 mL), and triethylamine (50 mL) at 80° C. to 83° C. The reaction mixture was washed with water and the organic layer was recovered. The reaction mixture was treated with methanol (150 mL) and filtered. The solid obtained was dried under vacuum at 50° C. to 55° C. to obtain the title compound.

### Yield: 64 g.

XRD provided

### EXAMPLE 5

### Preparation of 5-{4-[4-(5-Cyano-1H-Indol-3-yl)Butyl]Piperazin-1-yl}-1-Benzofuran-2-Carboxamide

Potassium carbonate (5.64 g) was added to water (50 mL) and stirred for 10 minutes. 5-Piperazin-1-ylbenzofuran-2-carboxamide (10 g), 3-(4-chlorobutyl)indole-5-carbonitrile (10.45 g), and 1-propanol (50 mL) were added to the reaction mixture. The reaction mixture was refluxed at 85° C. to 90° C. and maintained for 11 hours. The reaction mixture was cooled to 50° C. Water (100 mL) and hydrochloric acid (4N; 40 mL) were added to the reaction mixture and filtered. Dichloromethane (150 mL), methanol (150 mL), and water (150 mL) were added to the reaction mixture. The reaction mixture was treated with activated carbon (1 g), filtered through celite, and washed with dichloromethane (50 mL) and water (50 mL). Triethylamine (10 mL) was added to the reaction mixture, the layers obtained were separated and the organic layer was recovered under vacuum. Methanol (20 mL) was added to the reaction mixture, stirred for 1.5 hours and filtered. The solid obtained was dried at 45° C. to 50° C. to obtain the title compound.

### Yield: 13 g.

### EXAMPLE 6

### Preparation of 5-{4-[4-(5-Cyano-1H-Indol-3-yl)Butyl]Piperazin-1-yl}-1-Benzofuran-2-Carboxamide

5-Piperazin-1-ylbenzofuran-2-carboxamide (2.0 g) was added to 3-(4-chlorobutyl)indole-5-carbonitrile (1.9 g) in dimethylformamide (20 mL). Potassium carbonate (1.2 g) was added to the reaction mixture and heated to 80° C. to 85° C. and maintained for 5 hours. The reaction mixture was cooled to 35° C. and water (50 mL) was added, stirred for 2 hours, and then filtered. The solid obtained was dried under vacuum at 45° C. to 50° C. to obtain the title compound.

### Yield: 1.0 g.

### EXAMPLE 7

### Preparation of 5-{4-[4-(5-Cyano-1H-Indol-3-yl)Butyl]Piperazin-1-yl}-1-Benzofuran-2-Carboxamide

Potassium carbonate (2.8 g) was added to water (25 mL) and stirred for 10 minutes. 5-Piperazin-1-ylbenzofuran-2-carboxamide (5.0 g), 3-(4-chlorobutyl)indole-5-carbonitrile (5.7 g), and toluene (25 mL) were added to the reaction mixture and heated to 90° C. to 95° C. for 29 hours. The reaction mixture was cooled to 30° C., filtered and washed with water (25 mL). The solid obtained was dried at 45° C. to 50° C. to obtain the title compound.

### Yield: 7.0 g.

### EXAMPLE 8

### Preparation of 5-{4-[4-(5-Cyano-1H-Indol-3-yl)Butyl]Piperazin-1-yl}-1-Benzofuran-2-Carboxamide Hydrochloride

5-{4-[4-(5-cyano-1H-indol-3-yl)butyl]piperazin-1-yl}-1-benzofuran-2-carboxamide (10.0 g) was added to 2-propanol (430 mL). The reaction mixture was heated to 80° C. to 83° C. The reaction mixture was filtered. The reaction mixture was heated to 70° C. to 80° C. and a solution of 2-propnaolic hydrochloride (0.1N; 230 mL) was added to the reaction mixture at 70° C. to 80° C. in 20 minutes. The reaction mixture was cooled to 25° C. to 30° C. and stirred for 2.5 hours. The solid obtained was filtered, washed with diethyl ether (30 mL) and dried under vacuum at 20° C. to 30° C. for 12 hours to obtain the title compound.

Yield: 9.9 g.

1. A process for the preparation of 5-{4-[4-(5-cyano-1H-indol-3-yl)butyl]piperazin-1-yl}-1-benzofuran-2-carboxamide of Formula I 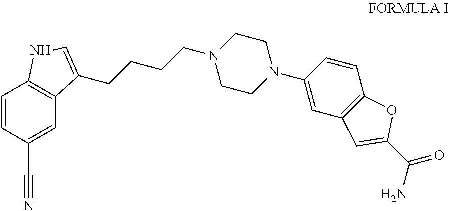 [SMILES: N#Cc1ccc2[nH]cc(CCCCN3CCN(c4ccc5oc(C(N)=O)cc5c4)CC3)c2c1]
or its pharmaceutically acceptable salts which comprises:
a) reacting 3-(4-chlorobutyl)-1H-indole-5-carbonitrile of Formula II 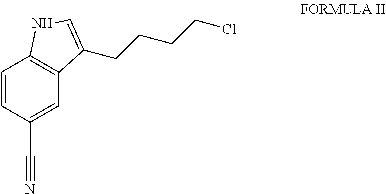 [SMILES: N#Cc1ccc2[nH]cc(CCCCCl)c2c1]
with 5-(piperazin-1-yl)-1-benzofuran-2-carboxamide of Formula III, 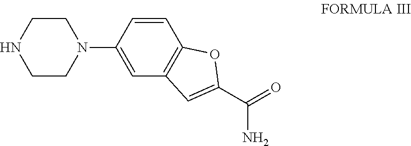 [SMILES: NC(=O)c1cc2cc(N3CCNCC3)ccc2o1]
in the presence of a base and a solvent;
b) isolating the compound of Formula I from the reaction mixture thereof; and
c) optionally converting the compound of Formula I to its pharmaceutically acceptable salts.

2. The process according to claim 1, wherein the base is selected from a group consisting of an organic base and an inorganic base.

3. The process according to claim 2, wherein the organic base is selected from triethylamine, diisopropylamine, diisopropylethylamine, 4-dimethylaminopyridine, pyrollidine, or N-methyl morpholine.

4. (canceled)

5. The process according to claim 2, wherein the inorganic base is selected from hydroxides or carbonates and bicarbonates of alkali or alkaline metals.

6. The process according to claim 5, wherein the carbonates and bicarbonates of alkali or alkaline metals are selected from sodium carbonate, potassium carbonate, magnesium carbonate, sodium bicarbonate, or potassium bicarbonate.

7. (canceled)

8. The process according to claim 1, wherein the solvent is selected from a group consisting of water, organic solvents, and a mixture thereof.

9. The process according to claim 6, wherein organic solvents are selected from a group consisting of alcohol, ketone, nitrile, amide, aromatic or aliphatic hydrocarbon, and dimethyl sulfoxide.

10. (canceled)

11. (canceled)

12. (canceled)

13. (canceled)

14. The process according to claim 1, wherein the solvent is water alone or in combination with 2-propanol, 1-propanol, dimethylformamide, or toluene.

15. The process according to claim 1, wherein the reaction of the compound of Formula II and the compound of Formula III is carried out in the presence of only water without using any other solvent.

16. The process according to claim 1, wherein the pharmaceutically acceptable salt is hydrochloride salt.

17. A crystalline form of vilazodone free base with substantially the same XRPD (X-Ray Powder Diffraction Pattern) as depicted in FIG. 1 or FIG. 2.

18. A crystalline form of vilazodone free base characterized by an XRPD having interplanar spacing (d) values substantially at 6.41, 5.14, 4.79, 4.43, 4.27, 4.17, 4.06, and 3.69±0.2 Å.

19. The crystalline form of vilazodone free base according to claim 18 which is further characterized by an XRPD having interplanar spacing (d) values substantially at 15.35, 12.05, 10.02, 8.07, 6.63, 6.41, 5.85, 5.55, 5.14, 4.79, 4.63, 4.43, 4.27, 4.17, 4.06, 3.85, 3.69, 3.54, 3.33, 3.20, 3.11, 3.03, 2.95, 2.89, 2.82, 2.70, 2.61, 2.52, and 2.32±0.2 Å.
